(19) United States
(12) Patent Application Publication
(10) Pub. No.: US 2007/0260277 A1
(43) Pub. Date: Nov. 8, 2007

(54) TUBULAR SHAFT INSTRUMENT

(75) Inventor: Josef Heinen, Nettetal (DE)

Correspondence Address: AKERMAN SENTERFITT, P.O. BOX 3188, WEST PALM BEACH, FL, 33402-3188, US

(73) Assignee: GIMMI GmbH, Tuttlingen (DE)

(21) Appl. No.: 11/743,553

(22) Filed: May 2, 2007

(30) Foreign Application Priority Data
May 2, 2006 (DE) 10 2006 020 594.4

(51) Int. Cl. A61B 17/00

(52) U.S. Cl. 606/205

(57) ABSTRACT

A tubular shaft instrument, in particular for the minimally invasive surgery, includes a shaft tube (10), an actuating element (12) axially displaceable in the shaft tube (10) and a grip provided at the proximal end of the tubular shaft instrument in the instrument axis. The grip includes two grip shanks (18), which are connected to each other at the proximal end, of which the free distal ends are spread apart by a spring force transverse to the instrument axis and can be pressed together against this spring force. At least one of the grip shanks (18) is connected with the actuating element (12) via a hinged or linked lever (20). On the opposite outer sides of the grip shank (18) finger rings (28) are provided on their distal end areas, which finger rings project outwards from the grip shanks (18).

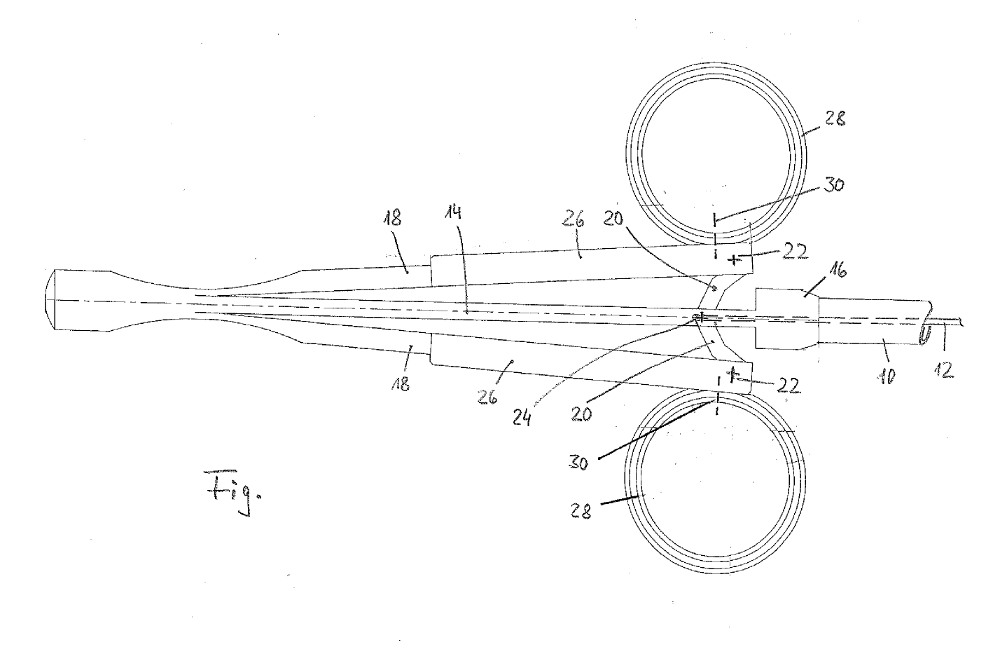

### FIELD OF THE INVENTION

The invention concerns a tubular shaft instrument, in particular for minimally invasive surgery.

### BACKGROUND OF THE INVENTION

Tubular shaft instruments of this generic type include a tubular shaft, comprising a shaft tube and a therein axially displaceable actuating element. At the distal end of the tubular shaft a work element is provided, which is comprised of two parts which are movable relative to each other by the axial movement of the actuating element in the shaft tube. Depending upon the function of the working elements and their coupling to the shaft tube and the actuating element, the actuating element could be a draw rod or a push rod.

In the proximal end of the tubular shaft a grip is provided, which includes two grip shanks, of which their movement relative to each other brings about an axial movement of the actuating element in the shaft tube.

In the known tubular shaft instruments in many cases the grip is in the form of a scissors grip, in which the grip shanks are provided angled relative to the instrument axis of the tubular shaft. Primarily for the use in access openings (trocar sheaths) with small diameter, tubular shaft instruments of the above-mentioned generic type are employed, in which the grip is provided essentially as the proximal axial extension of the tubular shaft. The grip shanks run parallel to, or at a very slight angle to, the instrument axis. The grip shanks are joined at the proximal end, while the distally oriented free ends of the grip shanks are urged apart by a spring force, so that they opening towards the distal direction at a slight angle relative to each other. For operating the instrument the grip shanks are pressed together against the spring force, wherein the pivot movement of the grip shanks are translated into an axial movement of the actuating element via a lever linked to the actuating element and the respective grip shank. One such design of the grip has the advantage, that the grip in its shape and operation essentially corresponds to a tweezers grip. The grip is grasped with two fingers, is thusly guided, and is operated by pressing together of the fingers. This allows a sensitive guidance and operation of the tubular shaft instrument and its distal work elements.

In general, by the pressing together of the grip shanks the work element is moved to its closed position, while a releasing of the grip shank leads to a moving apart of the grips shanks by the spring force, whereby the work elements are again moved back to their open position. If the work element is designed as a cutting element (for example as scissors or die) or as gripping element (for example as forceps or needle holders), then the work function is brought about by pressing together of the grip shanks, while the functionless opening movement is brought about by the spring force. If the opening movement of the work element must act against a resistance, for example against the tissue surrounding the distal tip of the tubular shaft instrument, then the opening movement brought about by the spring force could be impeded. In particular, this problem can occur when the tubular shaft instrument is in the form of a dissection forceps or retractor, in which case the work element is used to press the surrounding tissue apart during opening or to keep the tissue spread apart. In this embodiment the function of the work element is brought about by the spring force spreading apart the grip shanks, so that the operator has no influence on the function of the work element.

### SUMMARY

The invention is concerned with a task of improving a tubular shaft instrument of the above-described generic type in such a manner that the function is more reliable and, in certain cases, more multi-functional.

This task is inventively solved by a tubular shaft instrument as further described herein.

Advantageous embodiments of the invention are set forth herein.

In the inventive tubular shaft instrument, finger rings are provided on the outside of the grip shanks of the tweezers-like grip. The tubular shaft instrument can thus be operated in the conventional manner, in that the grip is grasped with two fingers resting on the grip shanks, which can press the grip shanks together against the force of the spring. The fingers operating the grip in accordance with the invention additionally engage in the finger rings provided on the grip shanks, so that the grip shanks can also be manually spread apart by the fingers of the operator. The operator can therewith, during spreading apart of the grip shanks, also manually augment the spring force. If a resistance occurs in particular during opening of the work elements, for example due to tissue lying against the work elements, which resistance cannot be overcome, or not completely overcome, by the spring force, then the operator can achieve a complete opening by the manual spreading apart of the grip shanks. This is advantageous for example when the tubular shaft instrument is used as retractor, in which a working element must be opened in order to separate tissue parts from each other and to keep these distanced.

In one advantageous embodiment the finger rings or at least one of the finger rings is provided rotatable on the respective grip shank. The finger rings can therewith be optimally adapted in their position with regard to the grip of the hand or the finger position of the operator, so that an ergonomically advantageous manipulation of the tubular shaft instrument is made possible.

### BRIEF DESCRIPTION OF THE DRAWING

In the following the invention will be described in greater detail on the basis of the illustrative embodiment shown in the FIGURE. The single FIGURE shows a side view of the grip of the tubular shaft instrument.

### DETAILED DESCRIPTION OF THE PREFERRED EMBODIMENTS

In the FIGURE only the grip of the tubular shaft instrument is shown. The tubular shaft with the shaft tube 10, the therein axially movable actuating element 12 and the work element located at the distal end of the tubular shaft are not shown. These parts are designed in conventional manner.

The grip is manufactured unitarily of metal, in particular of steel, and designed as a tweezers with rounded grip, which proximally joins the tubular shaft axially along the instrument axis.

The grip has a central base piece 14 which has the shape of a longitudinally extending flat cuboid and runs along the axis of the shaft tube 10. On the distal end of the central base piece 14 a coupling element 16 is formed, which serves for coupling of the shaft tube 10. As shown in dashed lines in the FIGURES, the proximal end of the actuating element 12 is axially free and movable through the coupling part 16 and engages in an axial longitudinal slit of the center piece 14 so that the proximal end of the actuating element 12 is also axially freely movable in the central base piece 14.

One grip shank 18 is provided respectively at the two flat sides of the center piece 14. The grip shanks 18 are connected unitarily at the proximal end to each other and to the center piece 14. The grip shanks 18 are so designed, that in their distal direction (in the drawing, to the right) oriented free ends are spread apart from the center piece 14, opening in the distal direction with a slight angle. The two grip shanks 18 are therein mirror symmetric to the center plane of the center piece 14. The elastic material characteristics of the grip shanks 18 make it possible to press together the free ends of the grip shanks 18 and thereby pivot them against their elastic spring force to lie against the center piece 14.

One lever 20 is linked respectively on the free ends of the grip shanks 18, with one lever free end pivotal about a rotation point 22. The other ends of the levers 20 are respectively pivotal about a rotation point 24 and linked to the proximal end of the actuating element 12. If the grip shanks 18 are pressed together against the elastic spring force, then the levers 20 pivot and their rotation point 24, together with the linked actuating element 12, is displaced in the proximal direction in the center piece 14. The actuating element 12 is thereby pulled back in the tubular shaft 10 in the proximal direction, whereby the not shown working element is actuated. If the grip shanks 18 are again released, they spread due to their spring force back into the position shown in the FIGURES, wherein the actuating element 12 is again pushed forwards via a lever 20 in the shaft tube 10 in the distal direction.

On the grip shanks 18 respectively one grip casing 26 is provided, which serve as finger rests.

Outside on the free distal ends of the grip shanks 18 respectively one finger ring 28 is provided. The finger rings 28 have, for example, the shape of a circular ring, in which each free inner grip opening makes possible the engagement of a finger, for example the thumb and index finger. The finger rings 28 are mounted to the grip shanks 18 rotatable about a rotation axis 30. The rotation axis 30 runs essentially in the plane of the opened grip shanks 18, within which the grip shanks 18 are pivotable, and transverse to the instrument axis, preferably perpendicular to the grip shanks 18. The finger rings 28 are so provided on the mount constituting the rotation axis 30 that the rotation axis 30 runs in the ring plane of the finger rings 28 and preferably runs diametrically through the finger rings 28.

The finger rings 28 can therewith assume, independently from each other, any desired rotation position of the finger rings with reference to the pivot axis of the grip shanks 18. Thereby, it is possible for the operator to engage with two fingers in the finger rings 28, for example thumb and index finger, in any position in which his hand may grip.

It is further conceivable that the inventive arrangement of the finger rings be also employed in a grip in which the grip shanks are not formed unitarily, but rather are connected to each other via a proximal linkage. In this case the spring force spreading apart the grip shanks is not the elasticity of the grip shanks but rather a supplementally provided spring.

Further, it is conceivable that the grip shanks are not necessarily provided mirror symmetric to the instrument axis. For example, one grip shank can be provided rigidly connected to the instrument axis and serve for coupling for the tubular shaft, while the other grip shank is spread apart from the fixed grip shank and can be pressed thereagainst. In this case only one grip shank is pivotable and this pivotable grip shank displaces the actuating element via a linked lever.

### REFERENCE NUMBER LIST

10 Shaft tube

12 Actuating Element

14 Center Piece

16 Coupling Element

18 Grip Shank

20 Lever

22 Rotation Point

24 Rotation Point

26 Grip Shell

28 Finger Ring

30 Rotation Axis

1. Tubular shaft instrument, in particular for minimally invasive surgery, comprising:
a shaft tube (10);
an actuating element (12) axially displaceable within the shaft tube (10); and
a grip provided at the proximal end of the tubular shaft instrument in an axis of the instrument, the grip including two grip shanks (18), which are connected to each other at the proximal end, of which free distal ends are spread apart by a spring force transverse to the instrument axis and can be pressed together against the spring force;
wherein at least one lever (20) linked at one lever end to a grip shank (18) and on a other lever end to the actuating element (12) translates the spreading movement of the grip shank (18) into an axial movement of the actuating element (12),
wherein on opposite outer sides of the grip shanks (18), on the distal end areas, respectively one finger ring (28) is provided, which extends outward from the grip shanks (18).

2. The tubular shaft instrument according to claim 1, wherein the finger rings (28) are oriented diametrically to each other with reference to the instrument axis.

3. The tubular shaft instrument according to claim 1, wherein the finger rings (28) are provided in the same axial position along the instrument axis.

4. The tubular shaft instrument according to claim 1, wherein at least one finger ring (28) is provided rotatable about a rotation axis (30) on the respective grip shank (18), which axis runs in a pivot plane of the grip shank (18) and essentially perpendicular to the respective grip shank (18).

5. The tubular shaft instrument according to claim 1, wherein the finger rings (28) are substantially circular ringed shaped.

6. Tubular shaft instrument according to claim 4, wherein the rotation axis (30) runs diametric to the finger ring (28).

7. The tubular shaft instrument according to claim 1, wherein the grip is a tweezers type rounded grip, of which the grip shanks (18) are provided connected unitarily to each other at the proximal end, and wherein the material elasticity of the grip shanks (18) provides the spreading spring force.

8. The tubular shaft instrument according to claim 1, wherein two finger rings (28) are provided rotatable about a rotation axis (30) on the respective grip shank (18), which axis runs in the pivot plane of the grip shank (18) and essentially perpendicular to the respective grip shank (18).
